(19) United States
(12) Patent Application Publication
(10) Pub. No.: US 2005/0004084 A1
(43) Pub. Date: Jan. 6, 2005

(54) Aspirin-containing trasdermal pharmaceutical composition for the treatment of calcification

(76) Inventor: Zoltan Dardai, Budapest (HU)

Correspondence Address: SPECKMAN LAW GROUP PLLC, 1501 WESTERN AVE, SEATTLE, WA, 98101, US

(21) Appl. No.: 10/495,778

(22) PCT Filed: Oct. 31, 2002
(86) PCT No.: PCT/HU02/00112

(30) Foreign Application Priority Data
Nov. 16, 2001 (HU) P 0104968

(51) Int. Cl. A61K 31/60; A61K 9/20

(52) U.S. Cl. 514/165; 424/465

(57) ABSTRACT

The invention relates to transdermally applicable aspirin-containing pharmaceutical compositions which can be used in the therapy of calcification. The compositions according to the invention also comprise for 1 g of acetylsalicylic acid an alkali metal carbonate, ammonium carbonate, an alkali metal hydrocarbonate and/or ammonium hydrocarbonate in an amount equivalent to 0.01-1 g of carbon dioxide, and are buffered to pH 5.5 to 7.

### FIELD OF THE INVENTION

The invention relates to transdermally applicable aspirin-containing pharmaceutical compositions which may be used in the therapy of calcification.

### BACKGROUND OF THE INVENTION

Calcification of cartilages, joints and interior organs is a disorder which affects many people in their lifetime, and plays a causative role in the development of numerous diseases or medical conditions (such as joint inflammations, rheuma, locomotor diseases, deformations, etc.). Numerous therapeutic methods are known for the treatment of calcification and its effects. These methods include n on-drug treatments (such as diet, curative gymnastics, massage, mud pack, balneotherapy, etc.) and various drug therapies (such as administration of antiphlogistic, analgesic, muscle-relaxant etc. agents), which are frequently used in combination. An important example of analgesic and antiphlogistic agents is acetylsalicylic acid (aspirin), which is used primarily for the treatment of rheumatic arthritis, rheumatoid arthritis, and arthritis-like conditions. Acetylsalicylic acid is usually administered in oral form or by injections, but it may also be administered transdermally. [Byoin Yakugaku 17(5), 335-340 (1991) (see C.A. 116, 158740q); Drugs of Today 33, 299-306 (1997); U.S. Pat. Nos. 4,275,059, 5,260,066 and 5,612,382; GB patent No. 1,036,314; French patent No. 2,295,753]. In addition to the above references, Arch. Int. Pharmacodyn. 177(1), 211-223 (1969) also indicates that acetylsalicylic acid possesses anti-calcification effects.

It is, however, well known that once acetylsalicylic acid enters the organism, it metabolizes within a short period of time and decomposes into salicylic acid and acetic acid as metabolites. Although these two metabolites are also effective in reducing established calcifications with the formation of calcium salicylate and calcium acetate [this effect of salicylic acid is discussed in Arch. Int. Pharmacodyn. 177 (1), 211-223 (1969), cited above], the formation of these two compounds, particularly calcium acetate, causes nephrological damages, due to its specific crystal structure. This may be the reason why acetylsalicylic acid has not been utilized in practice to stop or reduce calcification, despite its promising activity.

### SUMMARY OF THE INVENTION

It has been found that in a medium buffered to pH 5.5-7 and in the presence of an alkali metal, or ammonium carbonate, and/or hydrocarbonate, the metabolism of acetylsalicylic acid can be slowed down to such an extent that at the area of calcification the conversion of acetylsalicylic acid to calcium acetylsalicylate proceeds much faster than its metabolism to salicylic acid and acetic acid. Acetylsalicilyc acid, once converted to calcium acetylsalicylate, does not metabolize any further but is excreted from the body in unchanged form without causing nephrological damage. The present invention therefore provides a nephrologically harmless pharmaceutical composition suitable for the targeted treatment of calcification.

### DETAILED DESCRIPTION OF THE INVENTION

The present invention provides a transdermal pharmaceutical composition comprising acetylsalicylic acid, together with a carrier, a diluent and/or other auxiliary agent suitable for transdermal application. The composition according to the present invention also comprises for 1 g of acetylsalicylic acid, an alkali metal carbonate, ammonium carbonate, an alkali metal hydrocarbonate and/or an ammonium hydrocarbonate in an amount equivalent to 0.01-1 g of carbon dioxide, and the composition is buffered to pH 5.5 to 7. Taking into account the body's ion equilibrium, it is preferable to use sodium carbornate as the alkali metal carbonate or sodium hydrocarbonate as the hydrocarbonate.

The transdermal compositions according to the present invention may comprise acetylsalicylic acid in amounts usually applied in known compositions of this type. The compositions may contain generally 0.1-30% by weight, preferably 0.5-20% by weight, and more preferably 2-15% by weight of acetylsalicylic acid, relative to the total weight of the composition. However, as well known to one skilled in pharmacotechnology, the active agent content may also vary with this type of composition.

The compositions according to the present invention may also comprise the alkali metal carbonate, ammonium carbonate, alkali metal hydrocarbonate and/or ammonium carbonate in an amount equivalent to preferably 0.01-0.5 g, more preferably 0.02-0.3 g, and most preferably 0.02-0.2 g of carbon dioxide, for 1 g of acetylsalicylic acid.

The compositions according to the present invention have a pH of 5.5 to 7, preferably about 6.5. For aqueous compositions, such as emulsions, gels, creams etc., comprising at least 5% by weight of water, these figures represent the pH measured in the composition itself. For non-aqueous compositions, such as fatty creams or plasters carrying the pharmacons in a dried layer, these figures represent the pH of the composition when contacted with water (e.g. smeared onto wet skin surface or immersed into water). Any conventional non-toxic, pharmaceutically acceptable buffers can be used to adjust the pH of the composition to the prescribed value. Of these buffers, tertiary amines (such as triethanol amine), salts of tertiary amines, and quaternary ammonium salts are preferred.

The compositions according to the present invention may be administered in any form suitable for topical use, such as solutions, emulsions, suspensions, ointments, creams, lotions, shake-up mixtures, gels, plasters, etc. The compositions may further comprise carriers, diluents and/or other auxiliary agents conventionally applied in such compositions in addition to the acetylsalicylic acid active agent, the metabolism delaying agent (alkali metal or ammonium carbonate and/or hydrocarbonate), and the buffer. Examples of such carriers, diluents and other auxiliary agents include: ointment bases, such as vaseline and lanoline; solvents and liquid diluents, such as water, alcohols and glycerol; thickeners and gellifying agents, such as polyvinyl alcohol, polyvinyl acetate and polymeric cellulose derivatives; ionic and non-ionic tenzides; odourants; substances for adjusting osmotic pressure; colorants and dyestuffs. Preferred embodiments of the compositions comprise a thickener, optionally along with a stabilizer, since they may be applied in a relatively high local dose onto the area to be treated. The auxiliary agent should not react with acetylsalicylic acid.

The compositions may optionally comprise biologically active substances as activity-complementing agents, such as anti-phlogistic agents, analgesic agents, antirheumatic agents, muscle relaxants, local anaesthetics, pore dilatators and/or skin irritation alleviating agents. Examples of activity-complementing agents also include active agents utilized in conventional formulations for the topical treatment of joint and rheumatic pains, such as camphor, menthol, lidocain, diclofenac, snake venom extract, and the like. Preferably, the inventive compositions also comprise agents for dilating skin pores and/or vasopermeability increasing agents as activity-complementing substances, such as capsaicine, histamine, and cantharis extract.

If any of the components of the composition comprises alkali metal or ammonium ions, the required amount of alkali metal or ammonium carbonate and/or hydrocarbonate may be formed from this component by adding a calculated amount of carbon dioxide.

In order to treat calcification, the composition according to the present invention is applied onto the area of the body to be treated. The composition exerts its effect transdermally. If the composition is in a non-aqueous form, water is provided by wetting the area to be treated or by immersing a plaster containing the composition into water before application.

Further details of the invention are illustrated by the following non-limiting examples.

### EXAMPLE 1

Gels with the compositions given below were prepared by known conventional pharmacotechnological methods:

(A)

| | |
|---|---|
| Acetylsalicylic acid | 5 g |
| Diclofenac-Na | 4 g |
| Sodium hydrocarbonate | 3 g |
| Cetyl-trimethyl-ammonium bromide | 2 g |
| Ethyl-methyl-cellulose | 5 g |
| Ethanol | 25 g |
| Distilled water | 52 g |
| pH of the gel is 6.1. | |

(B)

| | |
|---|---|
| Acetylsalicylic acid | 10 g |
| Triethanol amine | 3 g |
| Lidocain | 3 g |
| Methyl cellulose | 4 g |
| Ammonium carbonate | 5 g |
| Ethanol | 30 g |
| Distilled water | 45 g |
| pH of the gel is 6.7. | |

(C)

| | |
|---|---|
| Acetylsalicylic acid | 10 g |
| Sodium carbonate | 2 g |
| Lidocain | 4 g |
| Tinctura capsaicini | 0.5 g |
| Triethanol amine | 3 g |
| Polyvinyl alcohol | 5 g |
| Ethanol | 22 g |
| Distilled water | 53.5 g |
| pH of the gel is 6.5. | |

### EXAMPLE 2

### Biological Activity Tests and Their Results

(A) Examination of the metabolism of acetylsalicylic acid

The tests were performed on 6 week old male New Zealand giant white rabbits. Before testing, the animals were subjected to radiography to ascertain that they did not suffer from calcification. The hair was shaved off from the backs of the rabbits, and 0.6 g of a gel with the following composition was applied onto the skin:

| | |
|---|---|
| acetylsalicylic acid | 0.05 g |
| ethanol | 0.05 g |
| methyl cellulose | 0.02 g |
| distilled water | 0.48 g |

Blood samples were taken periodically as indicated in Table 1 from the jugular veins of the animals, the samples were centrifuged to separate the plasma, and the acetylsalicylic acid (ASA) and salicylic acid (SA) contents of the samples were measured by HPLC. The centrifuged plasma was stored at −22° C. until the time of analysis in order to avoid any ex vivo metabolism due to the presence of esterases in the plasma. The results are summarized in Table 1.

TABLE 1
Salicylic acid and acetysalicylic acid contents
of blood samples, μg/ml of plasma

| Time | Rabbit 1 | | Rabbit 2 | | Rabbit 3 | | Rabbit 4 | |
|---|---|---|---|---|---|---|---|---|
| hours | ASA | SA | ASA | SA | ASA | SA | ASA | SA |
| 0 | 0 | BLD | 0 | BLD | 0 | BLD | 0 | BLD |
| 0.25 | 0.088 | 18.74 | BLD | 17.07 | 0.086 | 25.78 | 0.312 | 45.29 |
| 0.5 | 0.094 | 29.02 | 0.105 | 28.48 | 0.091 | 28.767 | 0.192 | 67.74 |
| 0.75 | 0.115 | 40.81 | BLD | 44.02 | 0.108 | 39.969 | 0.19 | 57.49 |
| 1 | 0.098 | 29.07 | 0.108 | 51.85 | 0.134 | 60.132 | 0.132 | 109.11 |
| 1.25 | 0.084 | 41.7 | 0.094 | 47.98 | 0.128 | 48.574 | 1.238 | 98.04 |
| 1.5 | 0.081 | 28.59 | 0.096 | 50.87 | BLD | 56.27 | 0.201 | 94.31 |
| 1.75 | 0.076 | 35.15 | BLD | 77.06 | BLD | 52.076 | 0.136 | 98.4 |
| 2 | BLD | 3 | BLD | 60.84 | BLD | 69.485 | 0.163 | 119.52 |

BLD = below the limit of detection

The data shown in Table 1 clearly demonstrate that the salicylic acid content of the blood samples was always at least one hundredfold of the acetylsalicylic acid content. The results of the measurements performed every 15 minutes verify that acetylsalicylic acid metabolizes in the body within 15 minutes to form salicylic acid and acetic acid.

The above test was repeated on the same animals with the difference that 0.01 g of sodium carbonate was also added to the gel and the gel was buffered to pH 6.5. The results are listed in Table 2.

TABLE 2
Salicylic acid and acetysalicylic acid contents
of blood samples, μg/ml of plasma

| Time | Rabbit 1 | | Rabbit 2 | | Rabbit 3 | | Rabbit 4 | |
|---|---|---|---|---|---|---|---|---|
| hour | ASA | SA | ASA | SA | ASA | SA | ASA | SA |
| 0 | 0 | 0 | 0 | 0 | 0 | 0 | 0 | 0 |
| 0.25 | 0.57 | 0.9 | 0.67 | 0.95 | 0.6 | 0.86 | 0.81 | 0.8 |
| 0.5 | 5.24 | 0.98 | 5.91 | 0.99 | 6.14 | 0.96 | 6.45 | 0.97 |
| 0.75 | 7.9 | 2.1 | 8.84 | 1.97 | 9.44 | 2.81 | 9.68 | 2.54 |
| 1 | 7.7 | 3.02 | 8.84 | 2.25 | 9.21 | 2.94 | 8.89 | 2.89 |
| 1.5 | 7.12 | 2.69 | 8.84 | 2.59 | 8.94 | 2.86 | 8.98 | 3.08 |
| 2 | 6.98 | 3.11 | 8.14 | 2.87 | 8.56 | 3.12 | 8.58 | 2.98 |
| 3 | 5.65 | 7.98 | 6.47 | 6.57 | 7.03 | 7.88 | 7.14 | 7.43 |
| 4 | 4.55 | 12.24 | 5.14 | 15.4 | 5.71 | 13.57 | 5.47 | 12.21 |

From the data of Table 2 it appears that the amount of acetylsalicylic acid present in the blood of the animals stabilized after a short induction period. An extreme slow down in the metabolism of acetylsalicylic acid was observed. Metabolism started essentially at about the third-fourth hour, however, 25% of acetylsalicylic acid remained un-metabolized even after 4 hours. This test was performed on healthy animals, where no dissolution of calcification and no formation of calcium acetylsalicylate due to the dissolution took place. As acetylsalicylic acid once reacted with the calcified area and thus converted to calcium acetylsalicylate, can no longer be metabolized but is excreted from the organism in unchanged form (which was verified by urine analyses performed on rabbits kept on specific calcium-rich diet), undesired calcium acetate formation can safely be avoided when a calcified organ is treated with the composition according to the present invention.

(B) Examination of calcification-dissolving effects under ex vivo conditions

The tests were performed on intact pig joints. One marble chip, about 5 mm in diameter and about 1-1.5 g in weight, was placed onto each of the joints, and the chips were smeared with a gel of the composition as given in point (A) of Example 1. After 8 hours, this treatment was repeated, and then the joints were allowed to stand overnight. Next day the effects were assessed visually. No marble residue could be observed on any of the joints. Upon subjecting the joints to surgical and radiographic examination, no damage of the bone, the synovial membrane, or the medullary substance could be detected either.

1-9. (cancelled)

10. A transdermal pharmaceutical composition comprising:
(a) acetylsalicylic acid; and
(b) at least one component selected from the group consisting of:
(i) an alkali metal carbonate,
(ii) ammonium carbonate,
(iii) an alkali metal hydrocarbonate, and
(iv) ammonium hydrocarbonate,
wherein, for each 1 g of acetylsalicylic acid, the at least one component is present in an amount equivalent to 0.01-1 g of carbon dioxide, and the composition has a pH of 5.5 to 7.

11. The composition of claim 10, further comprising at least one component selected from the group consisting of: carrier, diluents and auxiliary agents suitable for transdermal application.

12. The composition of claim 10, wherein the acetylsalicylic acid is present in an amount of 0.1-30% by weight.

13. The composition of claim 10, wherein for each 1 g of acetylsalicylic acid, the at least one component is present in an amount equivalent to 0.01-0.5 g of carbon dioxide.

14. The composition of claim 13, wherein for each 1 g of acetylsalicylic acid, the at least one component is present in an amount equivalent to 0.02-0.3 g of carbon dioxide.

15. The composition of claim 14, wherein for each 1 g of acetylsalicylic acid, the at least one component is present in an amount equivalent to 0.02-0.2 g of carbon dioxide.

16. The composition of claim 10, wherein the alkali metal carbonate is sodium carbonate.

17. The composition of claim 10, wherein the alkali metal hydrocarbonate is sodium hydrocarbonate.

18. The composition of claim 10, further comprising a buffer selected from the group consisting of: tertiary amines, tertiary amine salts, and quaternary ammonium salts.

19. The composition of claim 18, wherein the buffer is triethanol amine.

20. The composition of claim 10, wherein the composition further comprises at least one agent selected from the group consisting of: antiphlogistic agents, analgesic agents, antirheumatic agents, muscle relaxants, local anaesthetics, pore dilatators, and skin irritation alleviating agents.
